(19) United States
(12) Patent Application Publication
(10) Pub. No.: US 2010/0062999 A1
(43) Pub. Date: Mar. 11, 2010

(54) Composition extracted from shark cartilage marrow

(76) Inventor: Chun Yuan Chen, Taipei (TW)

Correspondence Address: BACON & THOMAS, PLLC, 625 SLATERS LANE, FOURTH FLOOR, ALEXANDRIA, VA, 22314-1176, US

(21) Appl. No.: 12/232,029

(22) Filed: Sep. 10, 2008

(51) Int. Cl. A61K 31/715; A61P 35/00

(52) U.S. Cl. 514/54

(57) ABSTRACT

The present invention relates to a composition extracted from shark cartilage marrow. This composition possesses effects of anti-melanoma effect, neutralization of gastric acid, and improvement of blood circulation and comprises Chondroitin Sulfate that is the active component thereof and is 50-60 wt % of the total weight of the composition. Besides, the composition has a molecular weight between 21-100 kilo Daltons and has energy state about 300-350 kilocalories per 100 grams.

### BACKGROUND OF THE INVENTION

1. Field of the Invention

The present invention relates to a shark cartilage extract and, more particularly, to a composition extracted from shark cartilage marrow.

2. Description of the Prior Art

In 1983, Drs. Lee and Langer published a paper in a scientific journal claiming that shark cartilage contains an angiogenesis inhibitor that can be used for inhibiting the vascularization of solid tumors and thus the proliferation of tumor cells. Since the paper was published, a surge of using shark cartilage as a treatment for tumors has been aroused. Drs. Lee and Langer extracted soluble components from fins of sharks by using 2M guanidine and directly applied the soluble components to tumors of animals. They found that neo-vascularization of tumors was inhibited and nutritional supply to tumor cells was cut, so that these tumor cells could be destroyed indirectly. After that, various reports concerning the anti-tumor effect of shark cartilage emerged. For example, a report claiming that injecting intravenously proteins extracted from shark cartilage can inhibit the proliferation of tumor cells. Another report claims that shark cartilage extraction can be used as anti-oxidants for elimination of reactive oxygen species. Another report claims that anti-tumor effect is found in mice after shark cartilage extract is planted. Besides, it is also found that shark cartilage extract has anti-inflammatory effect and can be used for inhibiting angiogenesis via oral administration. With these new pieces of evidence, shark cartilage seems to become a most promising treatment for tumors and numerous institutions including many universities and hospitals conduct lots of experiments and researches on shark cartilage. Although the effect of shark cartilage via oral administration is doubted, National Institutes of Health has conducted human clinical trial on shark cartilage since 1987 and the human clinical trial is in phase III now.

Conventionally, shark cartilage is usually smashed or ground into powders and the powders obtained by this way are characterized by strong fishy smell and gray or black color that will be unattractive to consumers. Besides, effective components in shark cartilage are usually unable to be extracted therefrom by this method.

In order to solve problems mentioned above, the inventor of the present invention has proposed a method disclosed in his United States Patent with the patent number U.S. Pat. No. 6,069,436 and the title “Method of obtaining extract of shark's cartilage”. As described in that patent specification, the method mainly comprises steps of crushing, washing, purifying, eliminating fishy smell, and sterilizing by heating up. Consequently, pure white extract without fishy smell can be obtained through the method for processing shark cartilage disclosed in that patent.

After that, the inventor proposes another new method for processing shark cartilage marrow based on above method to obtain highly purified shark cartilage marrow extract (SDS; shark distillated for saccharide). This method includes steps of enzyme dissolution, hydrolysis, homogenization, precipitation, and separation. The composition extracted from shark cartilage marrow according to the present invention is obtained by means of this method.

### SUMMARY OF THE INVENTION

An object of the present invention is to provide a composition comprising Chondroitin Sulfate as a principal component and having effects of anti-melanoma, neutralization of gastric acid, and improvement of blood circulation.

In order to achieve the above objects, the present invention provides a composition extracted from shark cartilage marrow comprising Chondroitin Sulfate that is 50-60 wt % of the total weight of the composition and is of molecular weight between 21-100 kilo Daltons.

In practice, the composition preferred comprises:
Chondroitin Sulfate, 56.3 wt % of the total weight of the composition;
total carbohydrates, 23.7 wt % of the total weight of the composition;
crude fat, 0.3 wt % of the total weight of the composition;
ash, 11.8 wt % of the total weight of the composition;
water content, 7.7 wt % of the total weight of the composition; and
sodium, 0.2 wt % of the total weight of the composition.

The following detailed description, given by way of examples or embodiments, will best be understood in conjunction with the accompanying drawings.

### BRIEF DESCRIPTION OF THE DRAWINGS

FIG. 1 shows a top view of a SDS/PAGE (sodium dodecyl sulfate polyacrylamide gel electrophoresis) of a composition according to the present invention.

FIG. 2 shows the relationship between the cumulative mortality of mice in experimental and control groups and time.

FIG. 3 is a bar diagram showing red and white blood cell contents of survived mice in experimental and control groups after the experiment is conducted.

FIGS. 4A and 4B are bar diagrams showing results of blood biochemical values of mice in experimental and control groups.

FIG. 5 shows dissection pictures of mice in experimental and control groups.

FIG. 6A shows the relationship between pH value and time in an in vitro experiment for swine gastric juice.

FIG. 6B shows the relationship between pH value and time in an in vitro experiment for neutralization of swine gastric juice.

FIG. 7A is a picture showing capillaries before the composition extracted from shark cartilage marrow according to the present invention is taken.

FIG. 7B is a picture showing capillaries after the composition extracted from shark cartilage marrow according to the present invention is taken.

FIG. 8A shows the result of blood flow velocity test for capillaries of scalp tissue from a Doppler ultrasound apparatus before the composition extracted from shark cartilage marrow according to the present invention is taken.

FIG. 8B shows the result of blood flow velocity test for capillaries of scalp tissue obtained from a Doppler ultrasound apparatus after the composition extracted from shark cartilage marrow according to the present invention is taken.

FIG. 9A shows the result of blood flow velocity test for genital capillaries obtained from a Doppler ultrasound apparatus before the composition extracted from shark cartilage marrow according to the present invention is taken.

FIG. 9B shows the result of blood flow velocity test for genital capillaries obtained from a Doppler ultrasound apparatus after the composition extracted from shark cartilage marrow according to the present invention is taken.

FIG. 10A shows the result of blood flow velocity test for mammary capillaries obtained from a Doppler ultrasound apparatus before the composition extracted from shark cartilage marrow according to the present invention is taken.

FIG. 10B shows the result of blood flow velocity test for mammary capillaries obtained from a Doppler ultrasound apparatus after the composition extracted from shark cartilage marrow according to the present invention is taken.

### DETAILED DESCRIPTION OF THE PREFERRED EMBODIMENTS

The present invention provides a composition extracted from shark cartilage marrow, comprising Chondroitin Sulfate that is 50-60 wt % of the total weight of the composition and being of molecular weight between 21-100 kilo Daltons. Besides, the composition is of energy state about 300-350 kilocalories per 100 grams.

Preferably, the Chondroitin Sulfate in the composition is 56.3 wt % of the total weight of the composition. Moreover, in addition to the Chondroitin Sulfate, the composition includes:
total carbohydrates, 23.7 wt % of the total weight of the composition;
crude fat, 0.3 wt % of the total weight of the composition;
ash, 11.8 wt % of the total weight of the composition;
water content, 7.7 wt % of the total weight of the composition; and
sodium, 0.2 wt % of the total weight of the composition.

Furthermore, the composition is white fine powder and is of energy state about 323 kilocalories per 100 grams.

Following are several experiments showing and proving the effects possessed by the composition extracted from shark cartilage marrow.

### Experiments

In this experiment, mice having melanoma are fed with the composition extracted from shark cartilage marrow (SDS; shark distillated for saccharide) according to the present invention. Then observe the growth of the melanoma in the abdominal cavities of the mice and estimate the anti-tumor effect of the composition. The method and steps thereof are described as follows.

### 1. The Proliferation and Purification of the Melanoma

The melanoma cell line (B16) is cultivated in Dulbelcco's Modified Eagle Medium (DMEM) with 2.5% Fetus Bovine Serum in an incubator that is provided with 5% CO₂. Add 1 ml 10% trypsin (Gibco) to digest cells that are cultivated at 90% confluency for 5 minutes and then take these cells out and count the cells.

### 2. Inducement of Tumor Cells in Abdominal Cavities of the Mice

Classify 80 four-week-old mice (BALB/C, purchased from Laboratory Animal Center, College of Medicine, National Taiwan University) into a control and a experimental groups. The mice in the control group are fed with routine feed while the mice in the experimental group are fed with feedstuff having 5% the composition extracted from shark cartilage marrow according to the present invention. After two weeks, inject 2.5×10⁶ melanoma cells into the abdominal cavities of the mice and count mortality of the mice after 14 days from the injection. Dissect dead mice and take out visceral organs after separating the tumor tissues. Then take photos of these visceral organs and compare them with each other. When the mortality exceeds half the mice, survived mice are sacrificed by cervical dislocation and blood samples of the sacrificed mice are collected for blood cell and blood biochemical analysis. The blood cell and blood biochemical analyses are undertaken after Heparin is added into the blood samples of the mice. The blood cell analysis is undertaken by using Sysmex SE9000 cell counter (Japan) while the blood biochemical analysis is undertaken by using Hitachi 7150. Finally, results are analyzed, calculated, and drawn by using Kaleidagraph.

### 3. Analysis of the Results

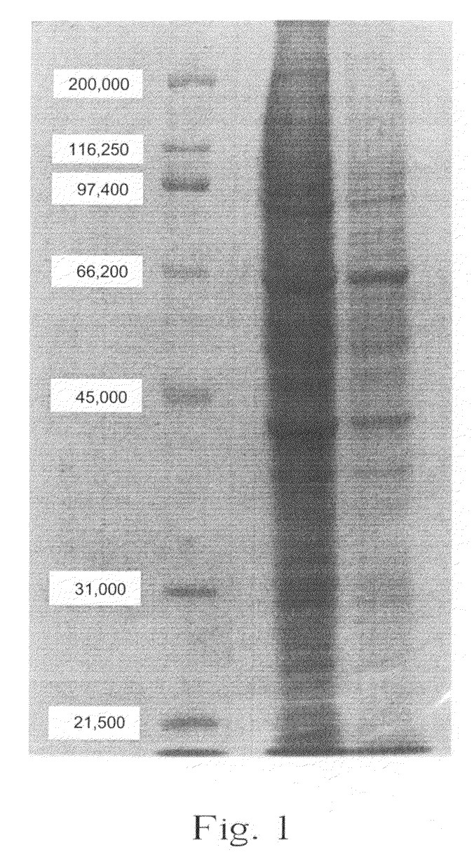

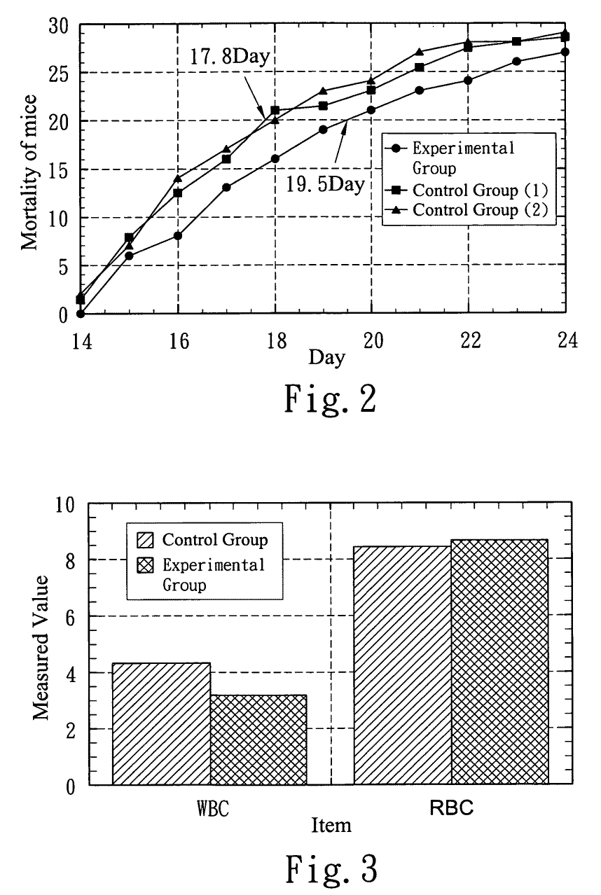

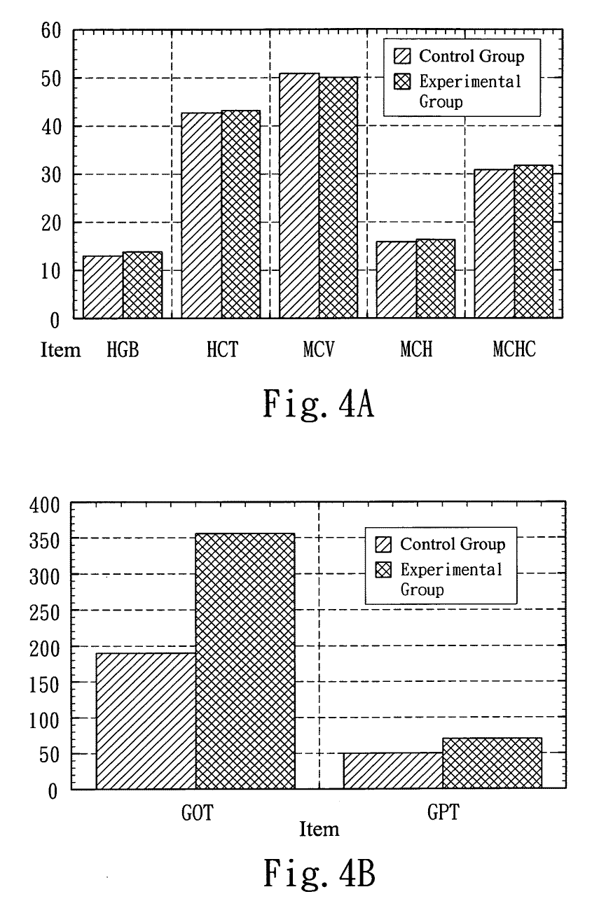

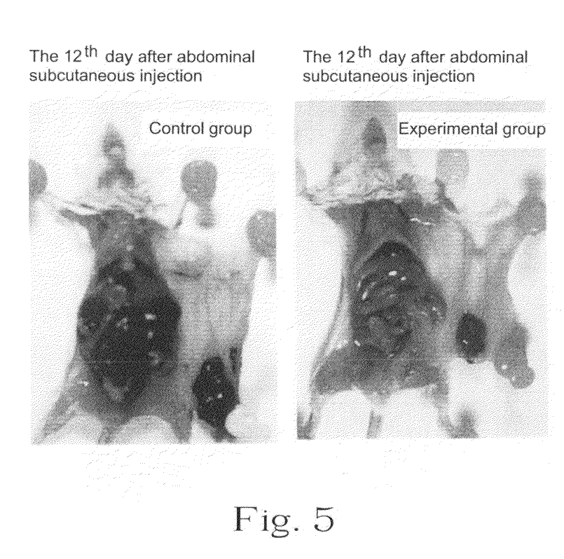

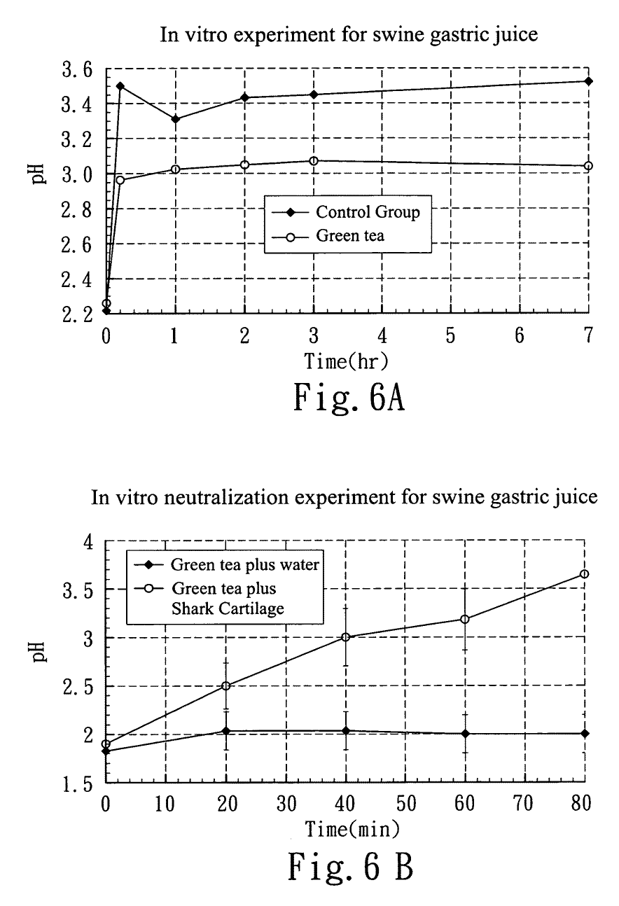

(1). Analysis of soluble components of shark cartilage by using SDS/PAGE (sodium dodecyl sulfate polyacrylamide gel electrophoresis):
   Please refer to FIG. 1. After the step of homogenization for processing shark cartilage, centrifuge the homogenized shark cartilage solution and collect the upper layer of the solution for SDS/PAGE. It is found that the main component of the upper layer of the solution is in the range of 21-100 kilo Daltons.
(2). The lifetime of the mice having ascites cancers can be significantly porlonged by administrating the composition extracted from shark cartilage marrow of the present invention.
   Forty mice in the experimental group are fed with feedstuff having 5% the composition extracted from shark cartilage marrow according to the present invention while 40 mice in each of two control groups are fed with routine feed. After two weeks, inject 2.5×10⁶ melanoma cells into the abdominal cavities of the mice. After 2-3 weeks, tumor tissues are formed as a result of great proliferation of the melanoma cells. These tumor tissues will metastasize to visceral organs and compress them. Accordingly, these compressed visceral organs of the mice will shrink gradually, blacken, and finally die of ischemia. Count the mortality of the mice each day and calculate and illustrate these results. The times where mortality exceeds half of the mice in the experimental group and in the control group are significantly different. As shown in FIG. 2, it averagely takes 19.5 days in the experimental group where the mice are fed with feedstuff having 5% the composition extracted from shark cartilage marrow while it averagely takes 17.8 days in the control group. Twenty-four days after injection of melanoma cells, the mortality exceeds half of the mice in each group. At this time, survived mice are treated with cervical dislocation and blood are collected for blood cell and blood biochemical analyses. The results of the blood cell analysis are shown in FIG. 3, where the numbers of white blood cells and red blood cells are not significantly different. The results of the blood biochemical analysis are shown in FIGS. 4A and 4B. As shown in FIG. 4A, the values of hemoglobin (HGB), hematocrit (HCT), mean corpuscular volume (MCV), mean corpuscular hemoglobin (MCH), and mean corpuscular hemoglobin concentration (MCHC) in the experimental and control groups are not significantly different. However, as shown in FIG. 4B, values of glutamic oxaloacetic transaminase/aspartate aminotransferase (GOT/AST) and glutamic pyruvic transaminase/alanine aminotransferase (GPT/ALT) are significantly different in the experimental and the control groups. In human blood biochemical analysis, the increase in both of the GOT/AST and GPT/ALT indicates hepatic injury. However, the normal range of the GOT/AST and GPT/ALT of BALB/C mice is unavailable and thus it is difficult to determine whether livers of the mice are impaired or the extent of hepatic impairment.
(3). Comparison of the tumor tissues with visceral organs:
   After dissect dead mice, compare the size and color of tumor tissues with visceral organs (hearts, kidneys, spleens, and livers) of the mice with each other. However, it is unable to distinguish differences of the size and color of tumor tissues and visceral organs with each other by naked eyes.
(4). Subcutaneous injection of melanoma cells:
   In this experiment of Subcutaneously injecting melanoma cells, mice in the experimental group fed with feedstuff having the composition extracted from shark cartilage marrow have longer survival time. The experiment is undertaken via abdominal subcutaneous injection of tumor cells and results thereof are assessed by distinguishing the proliferation differences of tumor tissues of the mice rather than by comparing the sizes of tumor tissues obtained after dead mice are dissected. Mice are fed with routine feeds and with feedstuff having 5% the composition extracted from shark cartilage marrow. After 12 days, abdominal subcutaneous inhection of 105 melanoma cells is undertaken. After two weeks from injection, these mice are treated with cervical dislocation and dissected. Dissection pictures of the mice are taken and the sizes of tumors are compared with each other. As shown in FIG. 5, compared with the tumor tissues of the mice fed with feedstuff having the composition extracted from shark cartilage marrow, the tumor tissues of the mice fed with routine feed are obviously larger and even metastasize into abdominal cavities. The tumor tissues of the mice in the experimental group are obviously smaller and do not metastasize into abdominal cavities. Accordingly, feeding mice with the composition extracted from shark cartilage marrow according to the present invention can inhibit the proliferation and the metastasis of subcutaneous melanoma cells of mice.
(5). The composition extracted from shark cartilage marrow can be used for neutralizing gastric juices.
   Mixed-breed pigs that are cross-breeding pigs between Landrace and LanYu small pigs and 3 months after weaning are used in this experiments. As shown in FIG. 6A, the pH value of the gastric juices from empty stomachs is 2.2. The pH value of the gastric juices will increase up to 3.4 after the gastric juices are mixed with the composition extract from shark cartilage marrow in vitro. Moreover, as shown in FIG. 6B, green tea is poured into the stomachs of the pigs in this in vivo experiment. Gather gastric juices are gathered every 20 minutes and pH values thereof are measured. The results reveal that the pH value decreases with time. However, the pH value increases with time in another in vivo experiment where the composition extracted from shark cartilage marrow in liquid form is poured into the stomachs of the pigs. Therefore, it can be obviously found that the decrease of the pH value as a result of green tea can be reversed by taking the composition extracted from shark cartilage marrow.

### 4. Discussion

Based on the results from above experiments, the proliferation and the metastasis of tumor cells can be inhibited and the lifetime of the mice injected with tumor cells can be prolonged after the composition extracted from shark cartilage marrow is giver orally. In conventional scientific literature, the effect of shark cartilage as an angiogenisis inhibitor is usually proved by having tumor tissues and shark cartilage extract in direct contact with each other via injection or surgery. Based on the results of above experiments, however, after the composition extracted from shark cartilage marrow is taken orally and digested and aborpted in the gastro-intestinal tract, the anti-tumor effect thereof still exists. Besides, it also can be found that the composition extracted from shark cartilage marrow has prominent effect for inhibiting over-secretion of gastric juices an thus digestive system can be protected.

Moreover, the composition extracted from shark cartilage marrow according to the present invention also has effect of promoting blood circulation.

When the composition in cream form applied onto skin begins to work, capillaries of the human body will dilate and water channels of bioelectric current will be open. Consequently, blood flow velocity will increase and the blood circulation and metabolism can be promoted as well. Consequently, skin temperature will increase and superficial thermal reaction can be produced accordingly. When the superficial thermal reaction is produced, water and body fluid of the human body will be agitated and the kinetic energy of water molecules will increase. Accordingly, deep thermal reaction will be produced as a result of the collision and the resonance of water molecules with increased kinetic energy since 70% of the human body is composed of water.

Theoretically, deep thermal reaction can be produced only by continuous exercises to convert kinetic energy into thermal energy and consume body fat and metabolize it into carbohydrates for use. Accordingly, functional disorder resulted from poor blood circulation can be overcome via molecular resonance and deep thermal reaction together with various ways and various substances.

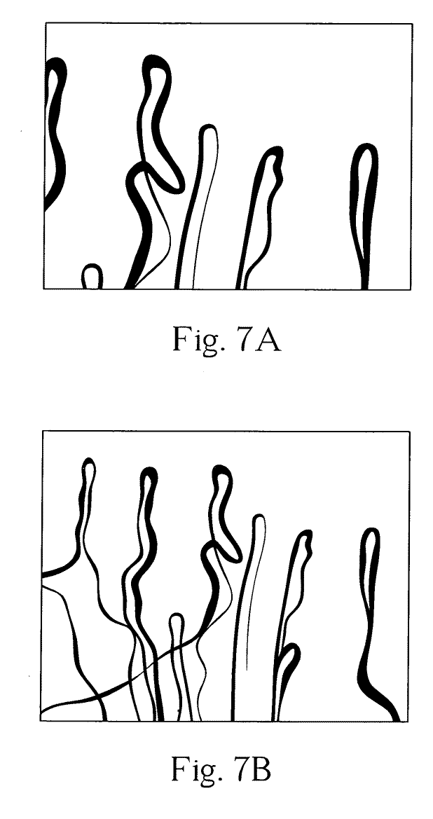

Please refer to FIGS. 7A and 7B that are pictures showing capillaries. The results of a capillary blood flow velocity test are shown in these figures. The pictures 7A and 7B are taken respectively before and after the composition extracted from shark cartilage marrow is taken. It can be found that capillary blood flow velocity can have a threefold increase by comparing above two pictures with each other after the composition of the present invention is taken.

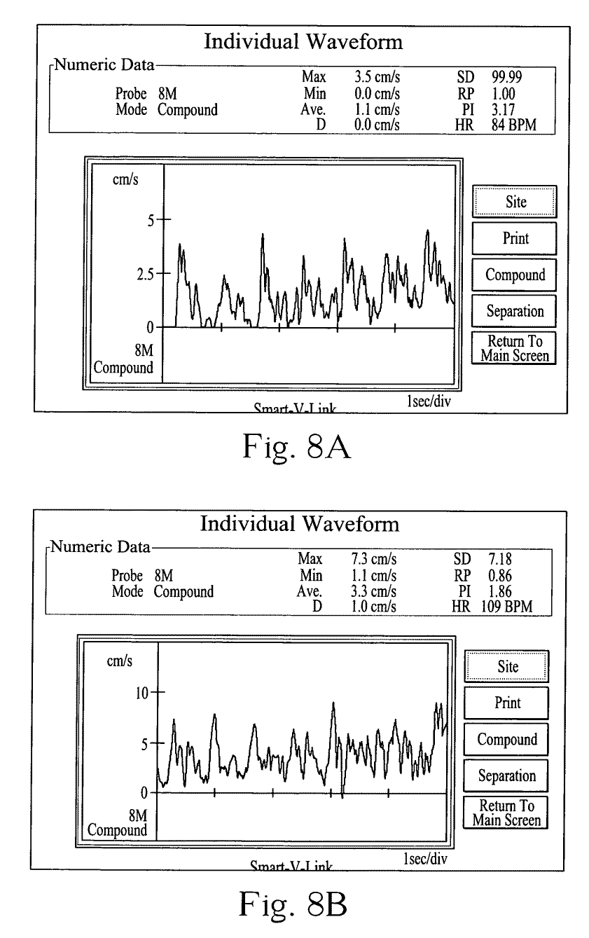

Moreover, FIGS. 8A and 8B show the results of a blood flow velocity test for capillaries of scalp tissue by using a Doppler ultrasound apparatus. As shown in these figures, capillary blood flow velocity is significantly increased after the composition according to the present invention is taken.

Based on this result, taking the composition according to the present invention is helpful to improve the blood circulation of scalp tissue and promote the metabolic rate thereof. As a result, the composition of the present invention can relieve the discomfort in head caused by poor blood circulation, strengthen scalp tissue able to resist pollutants, prevent from hair loss and aging, and provide hair follicles with sufficient nutrition for growing new hairs. Therefore, the composition of the present invention is effective for hair re-growth.

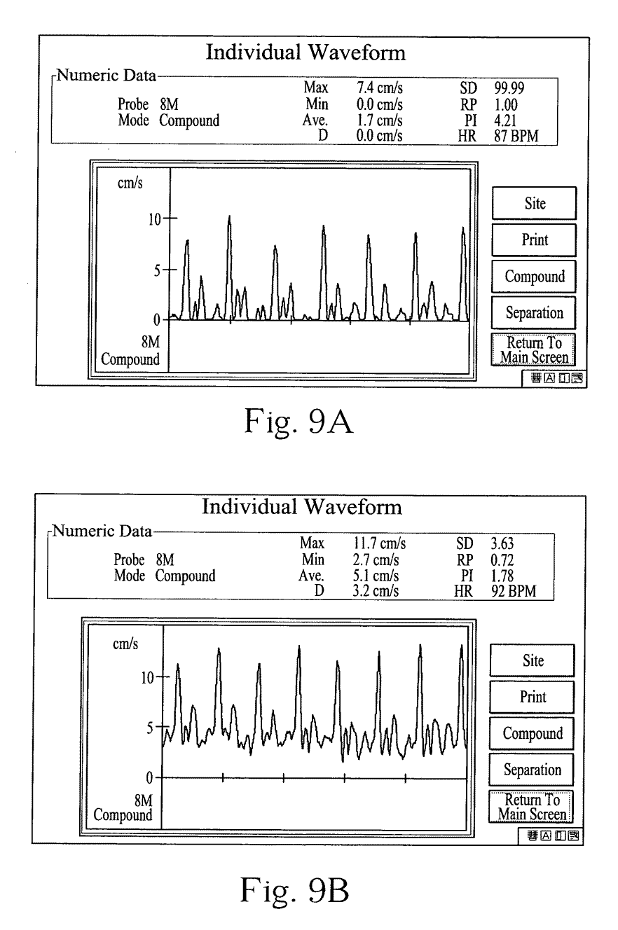

FIGS. 9A and 9B show the results of a blood flow velocity test for capillaries of genital organs by using a Doppler ultrasound apparatus. As shown in these figures, capillary blood flow velocity is significantly increased after the composition of the present invention is taken.

According to above results, the switch of bioelectric current can be turned on to restore the free transmission of signals of nervous plexus, so that patients can be recovered from impotence. Besides, increasing basal body temperature is also helpful to promote metabolic rate and capillary blood flow velocity will be increased accordingly. Capillary blood flow velocity can be increased by this way and it is advantageous for rapid perfusion of corpus cavernous penis. Moreover, female genital organs can be plumper, more elastic, and provided with more body fluid for lubrication as a result of the increased capillary blood flow velocity. In summary, the composition of the present invention is beneficial for overcoming the functional disorders of urogenital system and metabolic system and is effective for anti-aging and anti-degeneration.

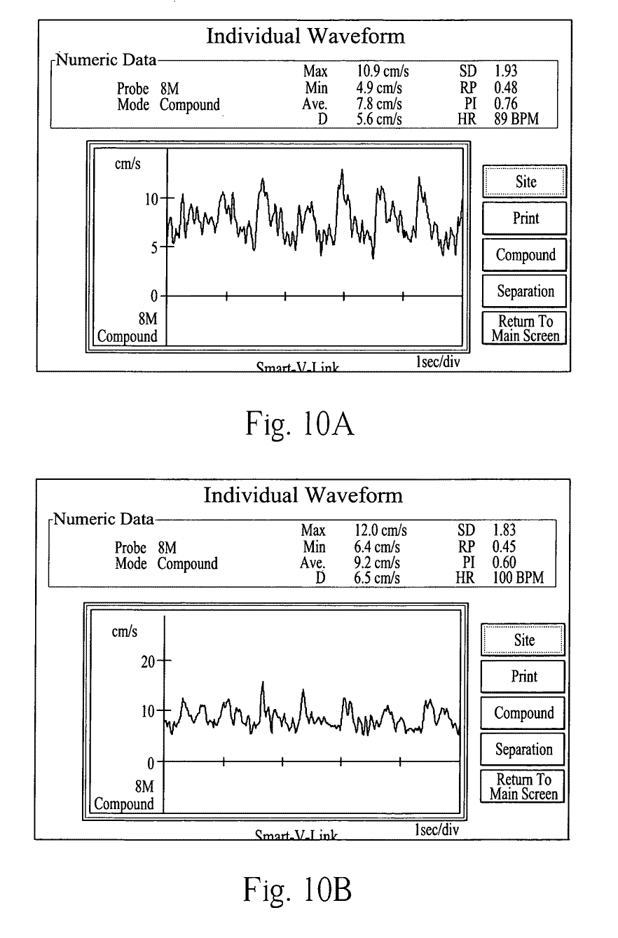

Please refer to FIGS. 10A and 10B showing the results of a blood flow velocity test for mammary capillaries by using a Doppler ultrasound apparatus. As shown in these figures, capillary blood flow velocity is significantly increased after the composition of the present invention is taken.

Based on above results, the composition of the present invention is able to increase the blood circulation to arouse molecular thermal reaction of tissue, so that the metabolic rate can be increased. By this way, exercise-like effect can be produced for improvement of mammary underdevelopment. The exercise-like effect is able to enlarge pectoralis major muscle for producing hormones that are able to assist in the second development of breast to make breast more upstanding.

As disclosed in the above description and attached drawings, the present invention can provide a composition extracted form shark cartilage marrow comprising Chondroitin Sulfate and having effects of anti-melanoma, neutralization of gastric acid, and improvement of blood circulation. It is new and can be put into industrial use.

Although the embodiments of the present invention have been described in detail, various modifications and variations may be made by those skilled in the art from the teachings disclosed hereinabove. Therefore, it should be understood that any modification and variation equivalent to the spirit of the present invention be regarded to fall into the scope defined by the appended claims.

What is claimed is:

1. A composition extracted from shark cartilage marrow, comprising Chondroitin Sulfate that is 50-60 wt % of the total weight of the composition and being of molecular weight between 21-100 kilo Daltons.

2. The composition extracted from shark cartilage marrow as claimed in claim 1, wherein the composition is of energy state about 300-350 kilocalories per 100 grams.

3. The composition extracted from shark cartilage marrow as claimed in claim 2, wherein the composition is of white fine powder.

4. The composition extracted from shark cartilage marrow as claimed in claim 1, wherein the Chondroitin Sulfate is 56.3 wt % of the total weight of the composition and the composition further includes:
total carbohydrates, 23.7 wt % of the total weight of the composition;
crude fat, 0.3 wt % of the total weight of the composition;
ash, 11.8 wt % of the total weight of the composition;
water content, 7.7 wt % of the total weight of the composition; and
sodium, 0.2 wt % of the total weight of the composition.

5. The composition extracted from shark cartilage marrow as claimed in claim 4, wherein the composition is of energy state about 323 kilocalories per 100 grams.

6. The composition extracted from shark cartilage marrow as claimed in claim 1, wherein the composition is of white fine powder.
